(19) United States
(12) Patent Application Publication
(10) Pub. No.: US 2020/0197646 A1
(43) Pub. Date: Jun. 25, 2020

(54) BI-MATERIAL SEALING ARRANGEMENT AND PATIENT INTERFACE DEVICE INCLUDING SAME

(71) Applicant: KONINKLIJKE PHILIPS N.V., Eindhoven (NL)

(72) Inventors: DANIEL STEED, MONROEVILLE, PA (US); DERRICK BLAKE ANDREWS, MONROEVILLE, PA (US); JAMES ROBERT MAIER, MONROEVILLE, PA (US); RICHARD ANDREW SOFRANKO, MONROEVILLE, PA (US)

(21) Appl. No.: 16/712,641

(22) Filed: Dec. 12, 2019

Related U.S. Application Data
(60) Provisional application No. 62/787,551, filed on Jan. 2, 2019.
(60) Provisional application No. 62/783,230, filed on Dec. 21, 2018.

(51) Int. Cl. A61M 16/06; A61M 35/00

(52) U.S. Cl. CPC A61M 16/0688; A61M 2210/0618; A61M 35/10; A61M 16/0622

(57) ABSTRACT

A sealing arrangement for use in delivering a flow of breathing gas to an airway of a patient includes a base structure having a surface positioned to engage the face of the patient. The base structure defines an opening having a perimeter which is sized and configured to be disposed about one or more of the mouth and/or nares of the patient. The sealing arrangement further includes a secondary element coupled to the base structure. The secondary element is positioned and structured to contact the face of the patient when the base structure is disposed with the perimeter about the one or more of the mouth and/or nares of the patient. The base structure is formed from a first material, and the secondary element is formed from a second material different than the first material.

### CROSS-REFERENCE TO RELATED APPLICATIONS

This patent application claims the priority benefit under 35 U.S.C. § 119(e) of U.S. Provisional Application No. 62/783,230, filed on Dec. 21, 2018, and 62/787,551 filed on Jan. 2, 2019, the contents of each which are herein incorporated by reference.

### BACKGROUND OF THE INVENTION

### 1. Field of the Invention

The present invention relates to non-invasive ventilation and pressure support systems wherein a patient interface device is used to deliver a flow of breathing gas to a patient and, more particularly, sealing elements for providing a seal between patient interface devices and subjects using such devices.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve a gas flow generator to produce a flow of breathing gas, and the placement of a patient interface device including a mask component on the face of a patient. The gas flow generator produces positive air pressure by taking air in from the surroundings and spinning a fan to push the air out of the machine, through a delivery conduit, and into the patient interface device to be delivered to the patient.

Traditional cushion members for patient interface devices include a sealing portion that is structured to engage the face of the patient in order to provide a seal therewith. Known sealing portions suffer from a number of drawbacks, such as applying too much pressure to certain areas of the face, resulting in red marks and a less than ideal fit. Another major problem is leaks. Stick-to-face adhesive masks could solve leaks, but in order to be reimbursable, cushions need to last for thirty days. It is difficult to create a stick-to face adhesive that works well for 30 days despite numerous reapplications and washings.

Furthermore, OSA patients tend to suffer from numerous comorbidities, some of which (e.g., hypertension (35% of OSA patients), diabetes mellitus type II (15% of OSA patients), depression (19% of OSA patients, etc.) are treatable by medication administered transdermally. Current CPAP masks do not provide for a secondary benefit, other than the increased health provided by CPAP therapy.

### SUMMARY OF THE INVENTION

Accordingly, it is an object of the present invention to provide an improved sealing arrangement for use in delivering a flow of a breathing gas to the airway of a patient.

As one aspect of the invention, such a sealing arrangement comprises: a base structure having a surface positioned to engage the face of the patient, the base structure defining an opening having a perimeter, the perimeter being sized and configured to be disposed about one or more of the mouth and/or nares of the patient; and a secondary element coupled to the base structure, the secondary element being positioned and structured to contact the face of the patient when the base structure is disposed with the perimeter about the one or more of the mouth and/or nares of the patient, wherein the base structure is formed from a first material, and wherein the secondary element is formed from a second material different than the first material.

The base structure may comprise a sealing flap of a cushion member of a patient interface device. The perimeter may be sized and configured to be disposed about both the mouth and the nares of the patient. The perimeter may be sized and configured to be disposed about only the nares of the patient. The base structure may comprise a nasal cushion. The second material may comprise a tacky material structured to adhere to the patient. The second material may comprise a transdermal drug delivery arrangement. The second material may comprise a material having a predetermined scent. The base structure may comprise a groove defined in the surface positioned to engage the face of the patient, and the secondary element may be coupled in the groove. The secondary element may be mechanically coupled in the groove. The secondary element may be coupled in the groove via a press-fit. The secondary element may be coupled in the groove via an adhesive. The secondary element may comprise a patient contacting surface, and the patient contacting surface may be flush with the surface of the base structure that is positioned to engage the face of the patient. The groove may extend entirely around the opening defined by the base structure. The groove may extend along only a portion of the perimeter. The secondary element may be coupled to the base structure via an adhesive. The secondary element may be coupled to the base structure via a mechanical coupling. The base structure may be overmolded onto the secondary element. The secondary element may be molded in the base structure. The secondary element may extend entirely around the opening defined by the base structure. The secondary element may extends along only a portion of the perimeter.

As another aspect of the invention, a secondary element for use with a base structure in a sealing arrangement for use in delivering a flow of breathing gas to an airway of a patient is provided. The secondary element comprises: a thin, elongate contoured member sized and configured to be disposed in a groove defined in a contoured patient contacting surface of the base structure.

The contoured member may comprise: a first contoured surface positioned to contact the patient and a second contoured surface disposed opposite the first surface; and an adhesive disposed on the second contoured surface. The contoured member may comprise a tacky material structured to adhere to the patient. The contoured member may comprise a transdermal drug delivery arrangement.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is an isometric view of a patient interface device and a portion of a conduit shown connected to a gas flow/pressure generating device (shown schematically) to form a system adapted to provide a regiment of respiratory therapy to a patient according to one exemplary embodiment of the invention;

FIG. 2 is rear elevation view of the cushion of the patient interface device of FIG. 1 showing a sealing arrangement in accordance with an example embodiment of the present invention;

FIG. 3 is an isometric view looking generally downward at the rear of the cushion of FIG. 2;

FIG. 4 is a sectional view of the cushion of FIG. 2 taken along line 4-4 of FIG. 2;

FIG. 5 is an enlarged view of a portion of the sectional view of FIG. 4 as indicated in FIG. 4; and

FIG. 6 is a rear elevation view of a cushion having another sealing arrangement in accordance with an example embodiment of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are coupled directly in contact with each other (i.e., touching). As used herein, “fixedly coupled” or “fixed” means that two components are coupled so as to move as one while maintaining a constant orientation relative to each other.

As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, left, right, upper, lower, front, back, on top of, and derivatives thereof, relate to the orientation of the elements shown in the drawings and are not limiting upon the claims unless expressly recited therein. As employed herein, the term “and/or” shall mean one or both of the elements separated by such term. For example, “A and/or B” would mean any of: i) A, ii) B, or iii) A and B.

As used herein, the phrase “mechanical bond” shall mean a bond formed as a result of the curing (i.e., solidifying) of a material selected from the group consisting of a monomer, a polymer, and a mixture of a monomer and a polymer (e.g., without limitation, silicone) to a fabric material. For example, without limitation, a bond formed when a viscous silicone material flows into fibers of a fabric material and is thereafter cured is a mechanical bond. A connection formed when a fabric material is stitched to a silicone material is not a mechanical bond.

As used herein, the phrase “chemical bond” shall mean a bond formed as a result of the curing (i.e., solidifying) of a first material to a second material, where each of the first and second materials is made of a monomer, a polymer, or a mixture of a monomer and a polymer.

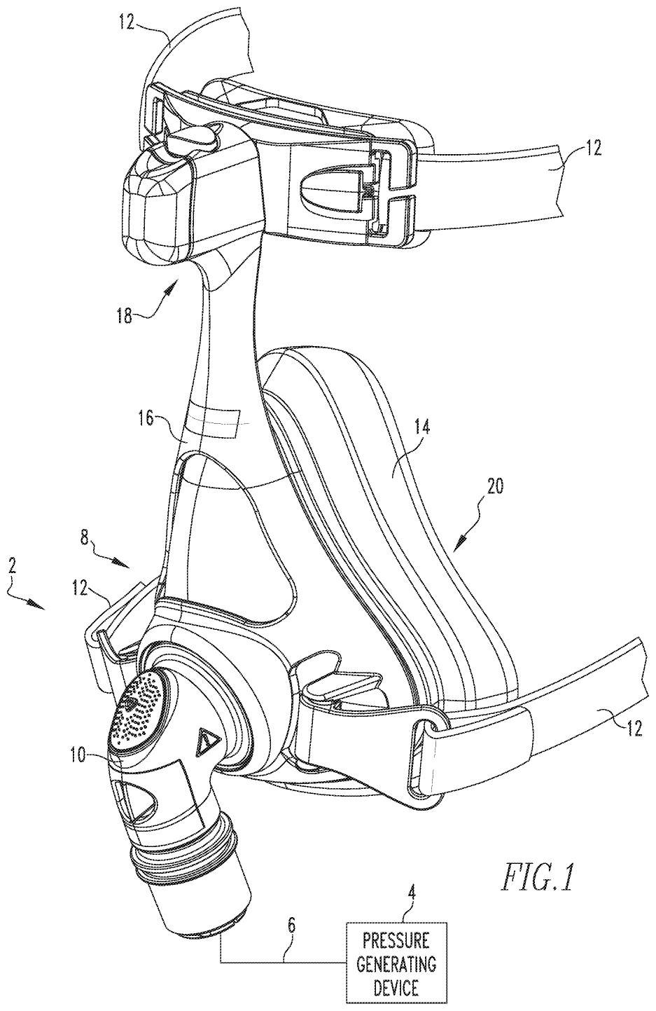

A system 2 adapted to provide a regimen of respiratory therapy to a patient according to one exemplary embodiment of the invention is generally shown in FIG. 1. System 2 includes a pressure generating device 4 (shown schematically), a delivery conduit 6 (shown schematically), a patient interface device 8 having a fluid coupling conduit 10, and a headgear 12 (only portions of straps thereof are shown). Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10. In the exemplary embodiment illustrated in FIG. 1, fluid coupling conduit 10 is an elbow connector, however, it is to be appreciated that other suitable couplings may be employed without varying from the scope of the present invention. Delivery conduit 6 and patient interface device 8 are often collectively referred to as a patient circuit.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, flow/pressure generating device 4 is also referred to as a gas flow generating device, because flow results when a pressure gradient is generated. The present invention contemplates that flow/pressure generating device 4 is any conventional system for delivering a flow of gas to an airway of a patient or for elevating a pressure of gas at an airway of the patient, including the pressure support systems summarized above and non-invasive ventilation systems.

In the exemplary embodiment illustrated in FIG. 1, patient interface device 8 is depicted as a full face mask which includes a generally pliable cushion 14 coupled to a generally rigid frame 16, both of which are coupled to conduit 6 via fluid coupling conduit 10. However, it is to be appreciated that other types of patient interface devices, such as, without limitation, an oral/nasal mask or a nasal cushion, which facilitates the delivery of the flow of breathing gas to the airway of the user, may be substituted for patient interface device 8 while remaining within the scope of the present invention. It is also to be appreciated that conduit 6 may be directly coupled to patient interface device 8 without the use of any intermediary coupling, such as conduit 10.

Cushion 14 may be formed of any pliable material (e.g., without limitation, silicone). Frame 16 may be formed of a substantially rigid material (e.g., without limitation, one or more plastics). Although frame 16 is shown having a forehead support 18, frame 16 is provided for exemplary purposes only and is not intended to be limiting upon the present invention as embodiments of the present invention are applicable to interface devices including various frame types as well as interface devices which do not utilize any frame.

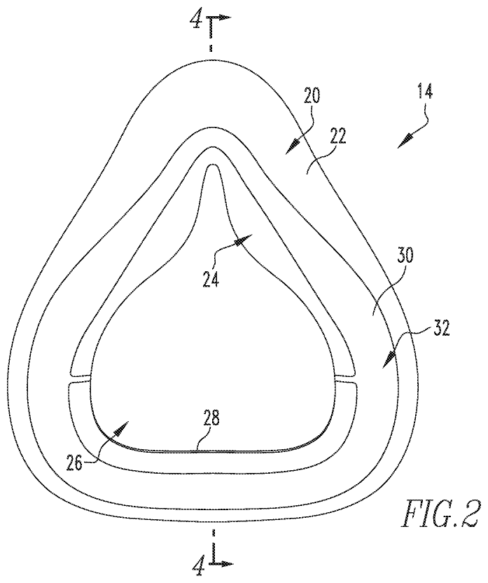

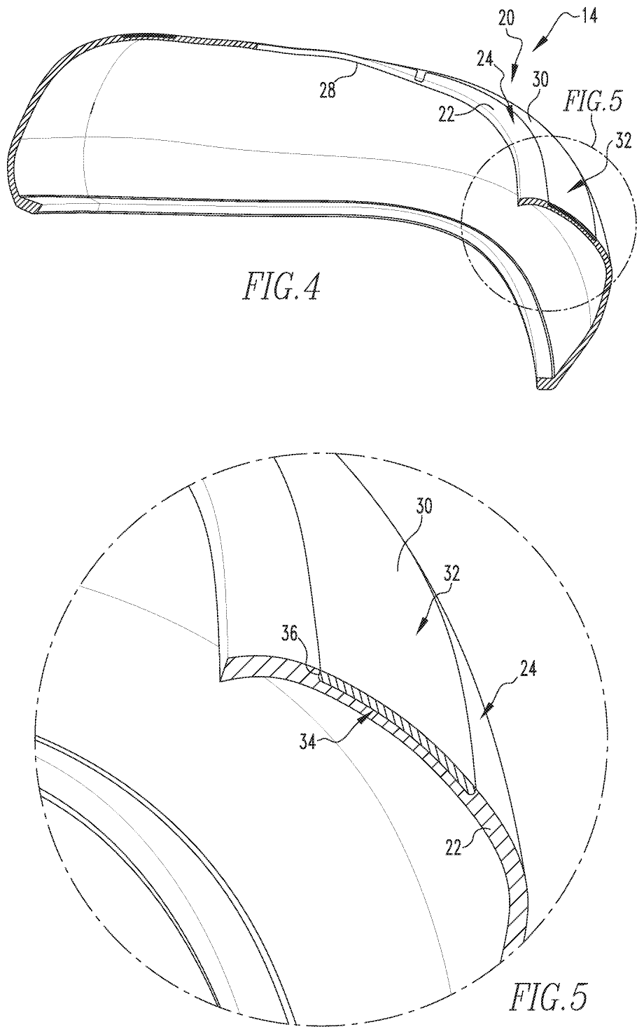

Referring to FIGS. 2-5, cushion 14 includes a sealing arrangement 20, in the form of a sealing flap. Sealing arrangement 20 includes a base structure 22 formed from a first material. In the example embodiment illustrated in FIGS. 1-4, base structure 22, is formed of silicone as a unitary portion of cushion 14. It is to be appreciated, however, that other suitable materials may be employed without varying from the scope of the present invention. Sealing arrangement 20 includes a contoured surface 24 positioned to engage the face of the patient when patient interface device 8 is disposed on the face of a patient. Base structure 22 defines an opening 26 having a perimeter 28 which is sized and configured to be disposed about one or more of the mouth and/or nares of the patient. In the example embodiment illustrated in FIGS. 1-5, perimeter 28 is sized and configured to be disposed about both the mouth and nares of a patient, however, it is to be appreciated that in the case of a nasal cushion perimeter 28 would be sized and configured to only surround the nares of the patient.

Continuing to refer to FIGS. 2-5, sealing arrangement 20 further includes a secondary element 30 formed from a different, or at least differing, material than base structure 22. Secondary element 30 is coupled to base structure 22 in a position such that secondary element 30 contacts the patient. Secondary element 30 is structured to provide a further benefit to the patient beyond that of base structure 22. For example, without limitation, secondary element 30 may be used to help adhere sealing arrangement 20 to the patient (thus reducing/eliminating the need for headgear 12) or to help reduce/eliminate leaks. In such instances, secondary element 30 would include an adhesive (e.g., an acrylic adhesive, an acrylate adhesive, or any other suitable adhesive) or other tacky material (e.g., ultra-low durometer silicone or thermoplastic elastomer (TPE)) positioned to contact the patient. As another example, secondary element 30 may be used to for transdermal delivery of a medication (e.g., topical medications such as antibiotics, corticosteroids to reduce inflammation, beauty-treatment regime, or any other treatment that may be applied in such area trans dermally) and thus may include a transdermal drug delivery arrangement. Secondary element 30 may also provide improved aesthetics (e.g., a particular color or colors, a printed or embedded pattern, etc.) or may comprise a material having a predetermined scent (e.g., lavender, vanilla, cinnamon, eucalyptus, menthol, etc.) which would appeal to the user and thus encourage adherence to a prescribed therapy.

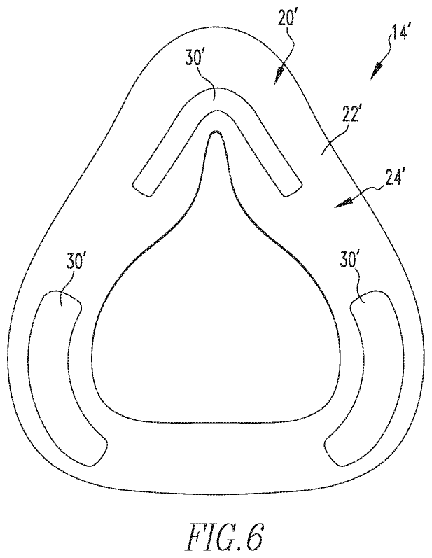

Secondary element 30 is formed as a thin (example embodiments are generally in the range of about 0.2 mm to about 2 mm), elongate contoured member having a first contoured surface 32 positioned to contact the patient and a second contoured surface 34 disposed opposite the first surface. In the example illustrated in FIGS. 2-5, secondary element 30 is sized and configured to be disposed in a groove 36 (FIG. 5) which is defined in contoured surface 24 of base structure 22. In example embodiments, secondary element 30 has been coupled in groove 36 via one or more of: mechanical coupling (e.g., a snap fit), press-fit, and adhesive. It is to be appreciated, however, that secondary element 30 may be coupled in groove 36 via any suitable means without varying from the scope of the present invention.

In the example embodiment illustrated in FIGS. 2-5, secondary element 30 is positioned such that first contoured surface 32 is disposed generally flush with contoured surface 24 of base structure 22. Such flush arrangement generally eliminates any potential marking to a patient's skin which may result in the transition between contoured surfaces 24 and 32. It is to be appreciated however, that some offset (e.g., preferably about 1 mm or less) between contoured surfaces 24 and 32 is acceptable without varying from the scope of the present invention. As an example, in other example embodiments of the present invention base structure 22 may not include groove 36 provided in contoured surface 24, in such embodiments secondary element 30 is coupled (e.g., via adhesive or suitable mechanical arrangement) on contoured surface 24, and thus not recessed into base structure 22.

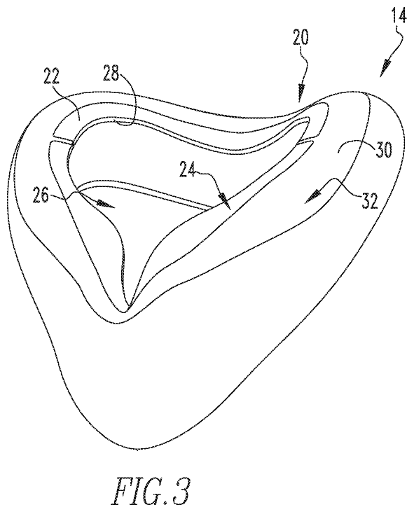

As shown in FIGS. 2 and 3, secondary element 30 may extend entirely around opening 26 defined by base structure 22. Alternatively, one or more secondary elements may be provided which each extend along only a portion of perimeter 28 of base structure 22. FIG. 6 shows a rear view, similar to that of FIG. 2, of an example of such an arrangement in accordance with an embodiment of the present invention in which a cushion 14′ includes a sealing arrangement 20′ having a plurality of secondary elements 30′ of differing shape and location coupled to a contoured surface 24′ of a base structure 22′.

It is to be appreciated that in addition to the coupling arrangements between base structure 22 and secondary element 30 previously discussed, other coupling arrangements may be employed without varying from the scope of the present invention. For example, for a more permanent arrangement, base structure 22 may be overmolded onto secondary element 30. Alternatively, secondary element 30 may be molded in base structure 22.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A sealing arrangement for use in delivering a flow of breathing gas to an airway of a patient, the sealing arrangement comprising:
a base structure having a surface positioned to engage the face of the patient, the base structure defining an opening having a perimeter, the perimeter being sized and configured to be disposed about one or more of the mouth and/or nares of the patient; and
a secondary element coupled to the base structure, the secondary element being positioned and structured to contact the face of the patient when the base structure is disposed with the perimeter about the one or more of the mouth and/or nares of the patient,
wherein the base structure is formed from a first material, and
wherein the secondary element is formed from a second material different than the first material.

2. The sealing arrangement of claim 1, wherein the base structure comprises a sealing flap of a cushion member of a patient interface device.

3. The sealing arrangement of claim 2, wherein the perimeter is sized and configured to be disposed about both the mouth and the nares of the patient.

4. The sealing arrangement of claim 2, wherein the perimeter is sized and configured to be disposed about only the nares of the patient.

5. The sealing arrangement of claim 1, wherein the base structure comprises a nasal cushion.

6. The sealing arrangement of claim 1, wherein the second material comprises a tacky material structured to adhere to the patient.

7. The sealing arrangement of claim 1, wherein the second material comprises a transdermal drug delivery arrangement.

8. The sealing arrangement of claim 1, wherein the second material comprises a material having a predetermined scent.

9. The sealing arrangement of claim 1, wherein the base structure comprises a groove defined in the surface positioned to engage the face of the patient, and wherein the secondary element is coupled in the groove.

10. The sealing arrangement of claim 1, wherein the secondary element is coupled to the base structure via an adhesive.

11. The sealing arrangement of claim 1, wherein the secondary element is coupled to the base structure via a mechanical coupling.

12. The sealing arrangement of claim 1, wherein the base structure is overmolded onto the secondary element.

13. The sealing arrangement of claim 1, wherein the secondary element is molded in the base structure.

14. The sealing arrangement of claim 1, wherein the secondary element extends entirely around the opening defined by the base structure.

15. The sealing arrangement of claim 1, wherein the secondary element extends along only a portion of the perimeter.
